(19) United States
(12) Patent Application Publication
(10) Pub. No.: US 2023/0000512 A1
(43) Pub. Date: Jan. 5, 2023

(54) MEDICAL DEVICE HANDLES WITH MULTIPLE DEGREES OF FREEDOM

(71) Applicant: Boston Scientific Medical Device Limited, Galway (IE)

(72) Inventors: Nabarun BHOWMICK, Kolkata (IN); Deepak Kumar SHARMA, Muzaffarnafar (IN); Shrikant Vasant RAUT, Mumbai (IN)

(73) Assignee: Boston Scientific Medical Device Limited, Galway (IE)

(21) Appl. No.: 17/852,986

(22) Filed: Jun. 29, 2022

Related U.S. Application Data
(60) Provisional application No. 63/216,650, filed on Jun. 30, 2021.

(51) Int. Cl. A61B 17/29

(52) U.S. Cl. CPC A61B 17/2909; A61B 2017/00424

(57) ABSTRACT

A medical device may comprise a handle having at least one actuator, a shaft having a proximal end and a distal end, the proximal end connected to the handle, and a distal assembly connected to the distal end of the shaft, the distal assembly including an end effector. The handle may be configured so that a single hand of a user can operate the at least one actuator to (1) actuate the end effector, (2) rotate the end effector relative to the shaft, and (3) articulate a distal portion of the shaft.

### CROSS-REFERENCE TO RELATED APPLICATIONS

This patent application claims the benefit under 35 U.S.C. § 119 to U.S. Provisional Patent Application No. 63/216,650, filed on Jun. 30, 2021, the entirety of which is incorporated herein by reference.

### TECHNICAL FIELD

Various embodiments of this disclosure relate generally to medical device handles. Examples of the disclosure relate to ergonomic handles that control multiple degrees of freedom of the medical device.

### BACKGROUND

In some medical procedures, a physician has to hold and manipulate multiple devices at a same time. For example, during endoluminal surgeries, a physician holds and manipulates a scope (e.g. an endoscope) with one hand (e.g. the left hand) while manipulating the scope shaft with the other hand (e.g. the right hand), to position the scope in a patient's body lumen. The physician or a technician then introduces an accessory device into a working channel of the scope and positions the accessory within the patient. Currently, accessories often do not have the capability to be manipulated independently and intuitively, in all degrees of freedom necessary for positioning and actuating the accessory. Additionally, manipulation of the scope and accessory device can lead to muscle fatigue and stress over the length of a procedure.

This disclosure is directed to overcoming one or more of these above-referenced challenges or other challenges in the art.

### SUMMARY

Aspects of the disclosure relate to, among other things, ergonomic medical device handles that allow for a single hand to control multiple degrees of freedom of the medical device in a neutral or relatively-neutral position. Each of the aspects disclosed herein may include one or more of the features described in connection with any of the other disclosed aspects.

According to certain aspects of the disclosure, a medical device may comprise a handle having at least one actuator, a shaft having a proximal end and a distal end, the proximal end connected to the handle, and a distal assembly connected to the distal end of the shaft. The distal assembly may comprise an end effector, wherein the handle is configured so that a single hand of a user can operate the at least one actuator to (1) actuate the end effector, (2) rotate the end effector relative to the shaft, and (3) articulate a distal portion of the shaft.

The medical device may include a first actuator, a second actuator, and a third actuator. The first actuator may rotate the end effector relative to the shaft, the second actuator may actuate the end effector, and the third actuator may articulate the distal portion of the shaft. The first actuator may be a trigger, the second actuator may be a knob, and the third actuator may be a knob, and the handle may be configured such that the user may simultaneously place an index finger of the single hand on the trigger, a thumb of the single hand on the first knob, a middle finger of the single hand on the second knob, and a palm of the single hand against the handle. The handle may be configured such that, in use, the first knob may be facing away from the user, the second knob may be on the left of the handle body relative to the user, and the trigger may be on the top of the handle. The medical device may include a mechanism to control the actuation of the end effector. The mechanism may include a first rack, a pinion gear meshed with the first rack, and a second rack meshed with the pinion gear and coupled to a control wire. When the first rack is moved into and out of a handle body of the handle, the pinion gear rotates clockwise and counterclockwise respectively, moving the second rack and the control wire proximally and distally respectively. A mechanism to control the rotation of the end effector relative to the shaft may include a first pinion gear, and a second pinion gear meshed with the first pinion gear. The control wire may be within the second pinion gear such that, when the first pinion gear is rotated clockwise and counterclockwise, the second pinion gear rotates counterclockwise and clockwise respectively, thereby rotating the control wire. A mechanism to control the articulation of the distal portion of the shaft may include a first control wire and a second control wire coupled to a pulley such that, when the pulley is rotated clockwise, the first control wire is pulled into tension and, when the pulley is rotated counterclockwise, the second control wire is pulled into tension.

According to another aspect of the disclosure, the first actuator may be a knob, the second actuator may be a trigger, and the third actuator may be a lever. The knob, the trigger, and the lever may be configured such that the user may simultaneously place an index finger of the single hand on the knob, a thumb of the single hand on the lever, a middle finger of the single hand on the trigger, and a palm of the single hand against the handle. The handle may be configured such that, in use, the knob is facing away from the user, the lever is facing towards the user, and the trigger is facing away from the user.

According to another aspect of the disclosure, the medical device may include a mechanism to control the actuation of the end effector that includes a first rack, a pinion gear meshed with the first rack, and a second rack meshed with the pinion gear and coupled to a control wire. When the first rack is moved when the first rack is moved into and out of a handle body of the handle, the pinion gear rotates clockwise and counterclockwise respectively, moving the second rack and the control wire proximally and distally respectively.

The medical device may include a mechanism to control the rotation of the end effector relative to the shaft. The mechanism may include a first pinion gear, a second pinion gear meshed with the first pinion gear and fixedly coupled to a proximal end of a shaft, a third pinion gear fixedly coupled to a distal end of the shaft, and a fourth pinion gear meshed with the third pinion gear, wherein the control wire is within the fourth pinion gear such that, when the first pinion gear is rotated clockwise and counterclockwise, the fourth pinion gear rotates clockwise and counterclockwise respectively, thereby rotating the control wire. Additionally, the medical device may include a mechanism to control the articulation of the distal portion of the shaft. The mechanism a first control wire and a second control wire coupled to a pulley such that, when the pulley is rotated clockwise, the first control wire is pulled into tension and, when the pulley is rotated counterclockwise, the second control wire is pulled into tension.

According to another aspect of the disclosure, the first actuator may be a knob, the second actuator may be a trigger, and the third actuator may be a lever. The knob, the trigger, and the lever may be configured such that the user may simultaneously place an index finger of the single hand on the knob, a thumb of the single hand on the lever, a middle finger of the single hand on the trigger, and a palm of the single hand against the handle. The handle may be configured such that, in use, the knob may be facing away from the user, the lever may be facing towards the user, and the trigger may be facing away from the user. The alternate device may include any combination of the previously described mechanisms to articulate, actuate, or rotate a distal portion of the shaft. Alternatively, the medical device may include a mechanism to control the rotation of the end effector relative to the shaft. The mechanism may include a first pinion gear, a second pinion gear meshed with the first pinion gear and fixedly coupled to a proximal end of a shaft, a third pinion gear fixedly coupled to a distal end of the shaft, and a fourth pinion gear meshed with the third pinion gear, wherein the control wire may be within the fourth pinion gear such that, when the first pinion gear is rotated clockwise and counterclockwise, the fourth pinion gear rotates clockwise and counterclockwise respectively, thereby rotating the control wire. Additionally, a mechanism to control the articulation of the distal portion of the shaft may include a first control wire and a second control wire coupled to a cam such that, when the cam is rotated clockwise, the first control wire is pulled into tension, and, when the cam is rotated counterclockwise, the second control wire is pulled into tension.

An alternate embodiment of the medical device may include at least one actuator. The one actuator may include a knob coupled to a base of the handle by a ball at the distal end of the knob and a socket on the proximal end of the base of the handle. The knob may be configured to include: a first mechanism to control the actuation of the end effector and including a control wire coupled to a distal end of the knob such that, when the knob is pulled proximally and pushed distally, the control wire is translated accordingly; a second mechanism to control the rotation of the end effector relative to the shaft and including the control wire coupled to a distal end of the knob such that, when the knob is rotated clockwise and counterclockwise, the control wire is rotated clockwise and counterclockwise, respectively; and a third mechanism to control the articulation of the distal portion of the shaft and including a first articulation wire and a second articulation wire coupled to the ball and configured such that, when the knob is moved in a first direction, the first articulation wire is pulled into tension, and, when the knob is moved in a second direction opposite the first direction, the second articulation wire is pulled into tension. The one actuator may be a knob coupled to the handle by a ball and a socket joint. The handle may be separable from the actuator.

Another aspect of this disclosure may include a method of operating a medical device. The method may include positioning the medical device inside a body lumen, articulating a distal portion of the shaft with at least one actuator using a single hand, rotating the end effector relative to the shaft with the at least one actuator using the single hand; and actuating the end effector with the at least one actuator using the single hand. The at least one actuator may include three actuators configured such that a user can simultaneously contact the three actuators at a same time using the single hand.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated herein and constitute a part of this specification, illustrate exemplary aspects of the disclosure and, together with the description, explain the principles of the disclosure.

FIG. 1 is a side view of a medical device, according to aspects of this disclosure;

FIGS. 2A and 2B are perspective views of a medical device, according to aspects of this disclosure;

FIG. 3 is a perspective view of a user holding the medical device of FIGS. 2A-2B, according to aspects of this disclosure;

FIG. 4 is a side cross-sectional view of the medical device of FIGS. 2A and 2B, according to aspects of this disclosure;

FIG. 5 is a side cross-sectional view of a magnified portion of the medical device of FIG. 4, according to aspects of this disclosure;

FIG. 6 is a side cross-sectional view of a portion of the medical device of FIG. 4, according to aspects of this disclosure;

FIGS. 7A and 7B are perspective views of an alternate embodiment of a medical device handle, according to aspects of this disclosure;

FIG. 8 is a perspective view of a user holding the medical device of FIGS. 7A and 7B, according to aspects of this disclosure;

FIG. 9 is a side cross-sectional view of the medical device of FIGS. 7A and 7B, according to aspects of this disclosure;

FIG. 10 is a perspective view of a user holding a medical scope and an alternate embodiment of a medical device, according to aspects of this disclosure;

FIG. 11 is a side view of the medical device of FIG. 10, according to aspects of this disclosure;

FIG. 12 is a side cross-sectional view of a portion of the handle of the medical device of FIG. 11, according to aspects of this disclosure;

FIG. 13 is a perspective view of a portion of the medical device of FIG. 11, according to aspects of this disclosure;

FIG. 14 is a side, partially cross-sectional view of portions of the medical device of FIG. 11, according to aspects of this disclosure;

FIGS. 15A and 15B are side, partially cross-sectional views of portions of the medical device of FIG. 11, according to aspects of this disclosure; and

FIGS. 16A, 16B, and 16C are perspective views of the alternate handle embodiments of the medical device of FIG. 11, according to aspects of this disclosure.

### DETAILED DESCRIPTION

Aspects of the disclosure include devices and methods to enable a neutral or relatively-neutral hand posture on a medical device handle (e.g. an ergonomic, natural hand position with the wrist and fingers generally at rest or near rest), with the handle controlling multiple degrees of freedom of the distal end of the device, to enable treatment at a target tissue site within a subject (e.g., patient). In embodiments, the handle is configured so that a single position of a hand on the handle may control up to five degrees of freedom of the device.

The medical device may be introduced into the body without a delivery device or via a delivery device. The delivery device may be a catheter, scope (endoscope, bronchoscope, colonoscope, etc.), tube, or sheath, inserted into a body cavity or lumen, for example the GI tract, via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, natural opening or body tract, or bodily incision.

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to devices and methods for performing various medical procedures and/or treating portions of the large intestine (colon), small intestine, cecum, esophagus, any other portion of the gastrointestinal tract, and/or any other suitable patient anatomy (collectively referred to herein as a “target treatment site”). Various examples described herein include single-use or disposable medical devices. Reference will now be made in detail to examples of the disclosure described above and illustrated in the accompanying drawings. Wherever possible, the same reference numbers will be used throughout the drawings to refer to the same or like parts.

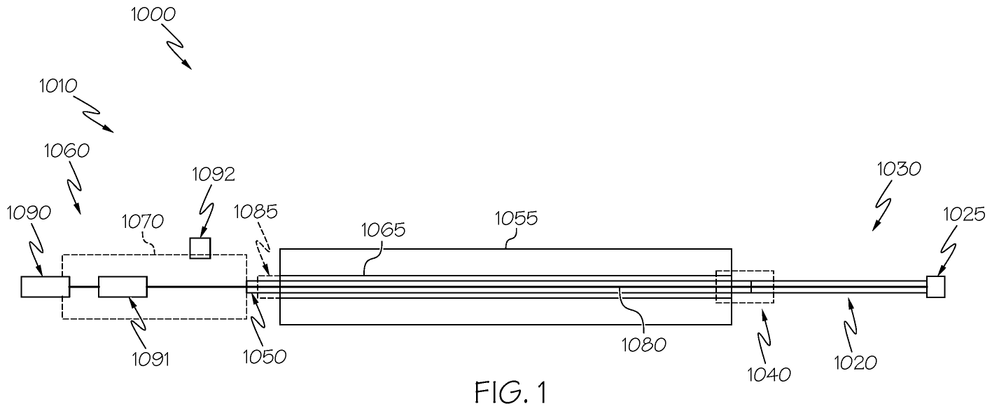

FIG. 1 is a general depiction of a medical system 1000 in accordance with examples of this disclosure. The system may be comprised of a medical device 1010 and a delivery device 1055. Medical device 1010 includes a proximal end 1060 and a distal end 1030. A handle 1070 including one or more actuators 1090, 1091, 1092 is at or adjacent to proximal end 1060. A shaft 1050 of device 1010 extends from a distal end of handle 1070 to the distal end 1030 of device 1010. Distal end 1030 includes a distal articulable section 1020 of shaft 1050 and an end effector 1025, to be described further therein.

Medical device 1010 may be introduced into the body via a delivery device 1055. Delivery device 1055 may include a port 1085 located at or adjacent to the proximal end of a lumen 1065 (e.g. a working channel) within delivery device 1055. Delivery device 1055 may be a catheter, scope (endoscope, bronchoscope, colonoscope, etc.), tube, sheath, or the like inserted into a body cavity or lumen, for example the GI tract via a natural orifice. Any structures of the medical devices described herein can be made of biocompatible materials, including biocompatible polymers, rubbers, plastics, and the like.

Still referring to FIG. 1, actuators 1090, 1091, 1092 of handle 1070 control various functions at distal end 1030. These actuators can include knobs, triggers, buttons, switches, pneumatic controls, or other actuators known in the art. Handle 1070 and actuators 1090, 1091, 1092 may enable multiple degrees of freedom of medical device 1010. For example, actuator 1090 may control the actuation (e.g. opening and closing) of end effector 1025; actuator 1091 may control the articulation (bending) of distal end 1030; and actuator 1092 may control the rotation of end effector 1025 relative to shaft 1050. Any combination of actuators and degrees of freedom are within embodiments of this disclosure. Additionally, medical device 1010 may be configured such that a user may rotate handle 1070 such that the entirety of medical device 1010 is rotated relative to delivery device 1055. Medical device 1010 may also be configured such that a user may be able to move handle 1070 back and forth such that medical device 1010 is moved relative to delivery device 1055, by translating within lumen 1065.

Shaft 1050 of medical device 1010 may be a tube having sufficient length to access sites within the body. Additionally, shaft 1050 may have sufficient flexibility to traverse tortuous anatomy. Shaft 1050 can be made of flexible materials, rigid materials, or any combination thereof.

Distal end 1030 is at or adjacent to the distal end of shaft 1050. Distal end 1030 is comprised of a distal articulable section 1020 of shaft 1050 (e.g. an articulation joint) and an end effector 1025. A transition zone 1040 is proximal to the distal articulable section 1020 of shaft 1050, and provides a transition between the articulable section and a more proximal portion of shaft 1050. Adhesives, ultrasonic welding, or any other means commonly known in the art may couple these components within transition zone 1040 (e.g. components at a proximal end of section 1020 to the more proximal portion of shaft 1050).

End effector 1025 can include a variety of components, including tools and parts to connect the tools to other parts of medical device 1010 and permit the various functions of those tools. Exemplary tools include, but are not limited to, a tissue grasper, a knife, biopsy forceps, scissors, a retrieval device (such as a net or a basket), an electrocautery tool, etc. As mentioned above, and as will be described in further detail below, various handle actuators (e.g. actuators 1090, 1091, and 1092) and related mechanisms can control the articulation of articulation section 1020 and the actuation (e.g., open/close movement) of end effector 1025. A connection between the actuators and the distal components, such as one or more elongate members 1080 (wires, cables, etc.), transmit the action of the actuators to the respective functionality at the distal end 1030.

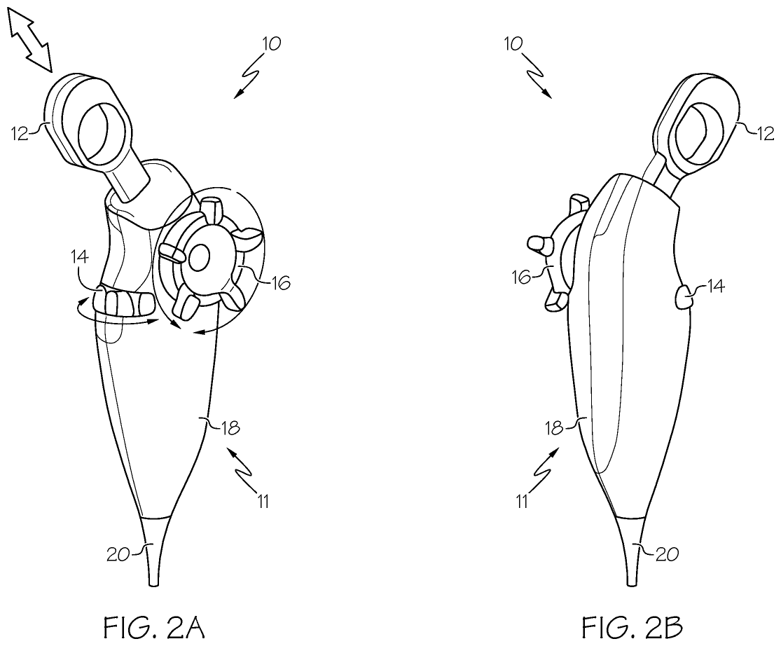

FIGS. 2A and 2B show an exemplary medical device 10 including a handle 11. Handle 11 includes a handle body 18 and three actuators: actuator/trigger 12 and knobs 14, 16. Strain relief 20 provides the junction between handle 11 and a shaft (not shown). Actuator 12 is on a proximal end of handle 11 and may control the actuation (e.g. open/close movement) of the end effector 1025, exemplified in FIG. 1. For example, moving actuator 12 away from handle body 18 can open (or otherwise actuate) the end effector (not shown), and moving the lever into handle body 18 can close (or otherwise actuate) the end effector. Alternate actions are also within the scope of this embodiment. For example, moving actuator 12 into handle body 18 (distally) can open the end effector, and moving actuator 12 away from handle 11 can close the end effector.

Knob 14 is located on a forward face of handle 11, e.g. a side of the handle facing away from the user when device 10 is in use. Knob 14 is perpendicular to a center axis of device 10 that extends from its shaft, through strain relief 20, and to a top of handle body 18 and proximate actuator 12. Knob 14 can control the rotational movement of the end effector 1025, exemplified in FIG. 1. For example, rotating knob 14 to the left can rotate the end effector in a counterclockwise motion, and rotating knob 14 to the right can rotate the end effector in a clockwise motion, or vice versa.

Knob 16 is located on a side face of handle 11, e.g. on the left side of the handle body 18 when device 10 is in use. A plane of rotation of knob 16 is parallel or substantially parallel to the center axis of device 10. Knob 16 can control the articulation direction of distal end 1030, exemplified in FIG. 1. For example, rotating knob 16 clockwise can result in the left articulation of the distal end, and rotating knob 16 in a counterclockwise motion can result in the right articulation of the distal end, or vice versa. The device can also be articulated up or down in similar fashion, depending on the attachment position of the steering wires to the distal end.

Handle 11 may contain any combination or subset of actuator 12 and knobs 14, 16, and each of actuator 12 and knobs 14, 16 can perform any of the various functions mentioned herein. For example, handle 11 may include only actuator 12 and rotation knob 14, or any other subset or combination. As another example, actuator 12 can control the articulation of distal end 1030 (shown in FIG. 1), knob 14 can control the actuation (e.g. open/close movement) of end effector 1025 (shown in FIG. 1), and knob 16 can control the rotation of distal end 1030 (shown in FIG. 1). Any combination thereof is within the scope of this disclosure.

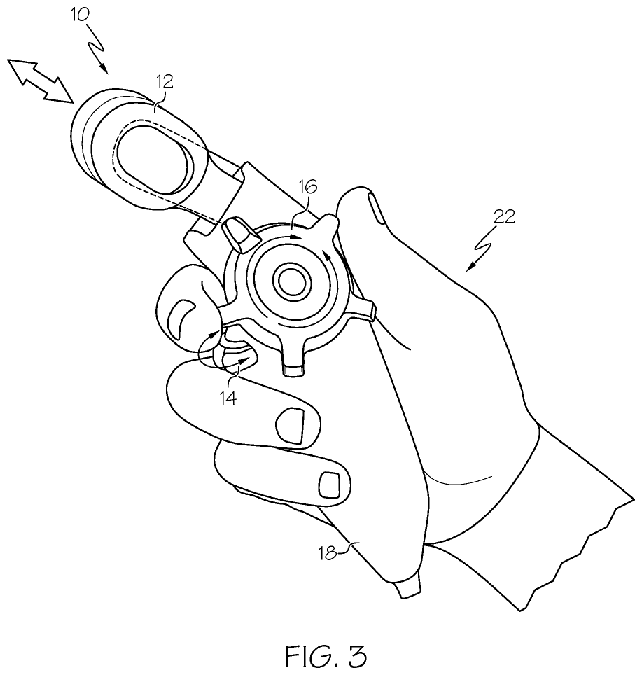

FIG. 3 shows how a user 22 may grip medical device 10, specifically handle 11, with a single hand. The thumb of user 22 may control clockwise and counterclockwise rotation of knob 16. The middle finger of user 22 may control the left and right rotation of knob 14. The index finger of user 22 may control the inward and outward movement of actuator 12. Actuator 12 is angled slightly away from the center axis of the device to enable a neutral, ergonomic grip for user 22. The remaining fingers and palm of user 22 may grip handle body 18. As shown, during use, knob 14 faces away from user 22, knob 16 is on the left side of handle body 18, and actuator 12 is at a top of handle body 18.

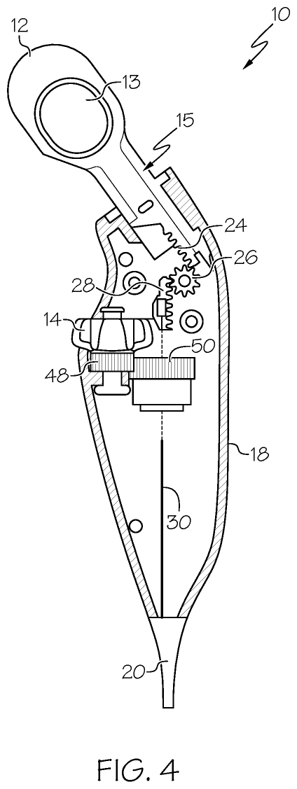

FIG. 4 shows a cross-section of handle 10 of FIGS. 2A and 2B, showing components that are interior of handle body 18. Actuator 12 includes a slot 13 and an extension 15. Slot 13 can be many shapes and sizes to enable a user to insert a finger through slot 13. For example, slot 13 can be ovular, as shown, circular, square, rectangular, etc. Extension 15 extends into handle body 18 and includes a linear rack 24 along the distal-most end of extension 15, opposite the end having slot 13. Rack 24 meshes with a pinion gear 26 such that, when actuator 12 is moved inward and outward of handle body 18 (through a slot in the top of handle body 18), pinion gear 26 rotates clockwise and counterclockwise, respectively. Pinion gear 26 meshes with a movable rack 28, and rack 28 is coupled to a control wire 30 in a manner that restricts movement of control wire 30 along its axis relative to rack 28, yet permits rotational movement of wire 30 relative to rack 28. As pinion gear 26 rotates, rack 28 moves up or down within handle body 18 (depending on the direction of rotation of pinion gear 26). And as rack moves up and down, wire 30 moves with it.

Still referring to FIG. 4, knob 14 is fixedly coupled to a pinion gear 48. Pinion gear 48 meshes with a pinion gear 50, and pinion gear 50, in turn, is coupled to control wire 30. Control wire 30 is coupled to gear 50 in a manner that allows wire 30 to translate up and down within gear 50, yet also rotate with gear 50. For example, a crimp having a square outer profile may be fixed to wire 30, between wire 30 and gear 50. Gear 50 may have a square shaped lumen at its center, to accept the square crimp. Rotation of gear 50 will therefore rotate the crimp and wire 30. When knob 14 is rotated, therefore, pinion gear 48 rotates in the same or similar rotational direction, causing the opposite rotation of pinion gear 50 and wire 30.

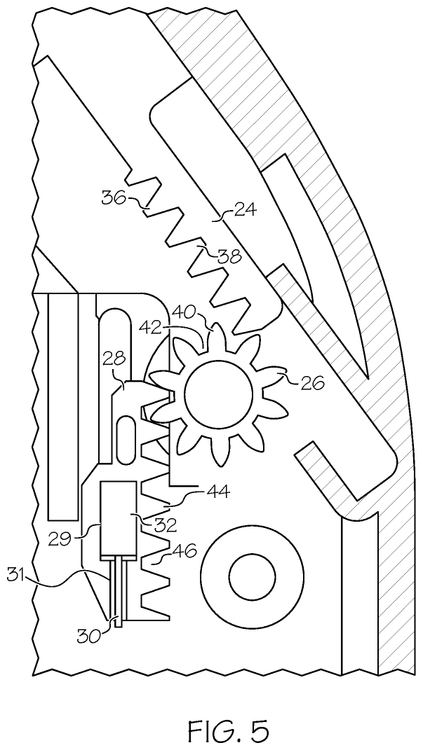

FIG. 5 shows a close-up of the interactions between rack 24, pinion gear 26, and rack 28. Rack 24 is comprised of a plurality of extensions 36 (e.g. teeth) and a plurality of indentions 38 (e.g. troughs) that mesh with a plurality of indentions 42 and extensions 40 on pinion gear 26, respectively. In addition, the plurality of extensions 40 and plurality of indentions 42 from pinion gear 26 mesh with the plurality of indentions 46 and plurality of extensions 44 of rack 28. Rack 28 may be comprised of two components that are mirror images of one another. FIG. 5 shows one of those components. The other component (not shown) would come out of the page of FIG. 5 and lay over the component shown to define a cavity in which a ferrule 32 sits. The two components of rack 28 may be coupled to completely or partially contain ferrule 32. Adhesives, ultrasonic welding, or any other means commonly known in the art may couple the components of rack 28. Ferrule 32 may be fixedly coupled to control wire 30 by a crimp, adhesive, or any other means commonly known in the art. Control wire 30 extends from the distal end of ferrule 32, through a channel 31 defined within rack 28. Channel 31 extends from cavity 29 to a distal end of rack 28. Control wire 30 then extends through handle body 18, strain relief 20, the shaft of device 10, to the distal tip of device 10. At the distal tip, wire 30 connects to a distal assembly in a manner that actuates an end effector of the distal assembly, in any suitable manner known in the art.

Rack 24, pinion gear 26, and rack 28 interact such that, when rack 24 is moved inward and outward of handle body 18, the plurality of indentions 38 and plurality of extensions 36 mesh with the plurality of extensions 40 and plurality of indentions 42 of pinion gear 26 to move it clockwise and counterclockwise, respectively. In turn, the plurality of extensions 40 and plurality of indentions 42 of pinion gear 26 mesh with the plurality of extensions 44 and plurality of indentions 46 of rack 28 such that, when pinion gear 26 is rotated clockwise or counterclockwise, the rack 28 is raised and lowered, respectively. When rack 28 is raised and lowered, ferrule 32 and control wire 30 are raised and lowered, accordingly. This movement correlates to the actuation of the distal assembly/end effector. Minor adjustments, including the addition of more pinion gears and racks, can be understood to enhance or change the desired movement. For example, the addition of another pinion gear between either rack 24 and pinion gear 26 or rack 28 and pinion gear 26 may result in an opposite action than that achieved with the arrangement shown in FIG. 5. More specifically, pushing rack 24 distally in FIG. 5 result in proximal movement of rack 28 to, for example, open an end effector. Adding another gear as described above would result in distal movement of rack 24 causing distal movement of rack 28 to, for example, close an end effector.

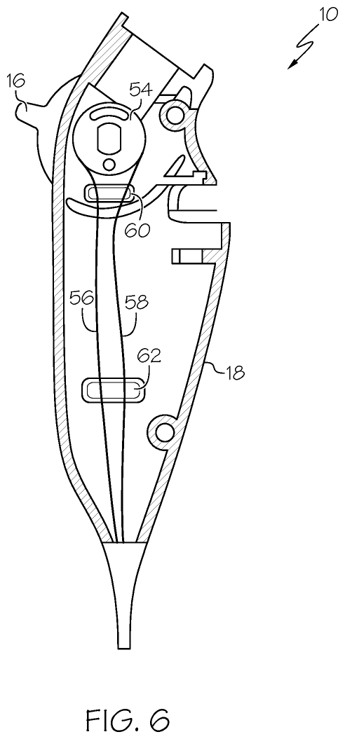

FIG. 6 shows an alternative cross-sectional view of medical device 10 of FIGS. 2A and 2B, showing components within handle body 18 that are not shown in FIG. 4. The components shown in FIG. 6 are used for articulating the distal end of the shaft (not shown) of the medical device. In FIG. 6, knob 16 may be fixedly coupled to a pulley 54 by press-fit, adhesives, or any method commonly known in the art. Steering wires 56, 58 may be coupled to pulley 54 such that, when knob 16 is rotated, one of steering wires 56, 58 is pulled into tension. Steering wires 56, 58 may be connected to the distal tip (e.g. a distal end of an articulation joint), such that, when a steering wire 56, 58 is pulled into tension, the distal tip (not shown) articulates accordingly. For example, rotating knob 16 counterclockwise may put tension on steering wire 56, resulting in the articulation of the distal tip in a first direction, while rotating knob 16 clockwise may put tension on steering wire 58, resulting in the articulation of the distal tip in a second direction opposite the first direction. Steering wires 56 and 58 may pass through tracks 60 and 62, which serve to confine the steering wires and prevent the wires from entangling with other components within the handle during use.

Aspects of the disclosure include methods of using device 10. To do so, the user may first introduce the distal end of device 10 into a GI tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any other natural opening or body tract, bodily incision, or through a delivery device, such as an endoscope or sheath. Once the desired site is accessed, the user can actuate one or more actuators, including knobs 14, 16 and actuator 12, with only one hand (or both hands if desired), to control the articulation of the distal end of the medical device, the actuation of the end effector, and/or the rotation of the end effector relative to the shaft of medical device 10.

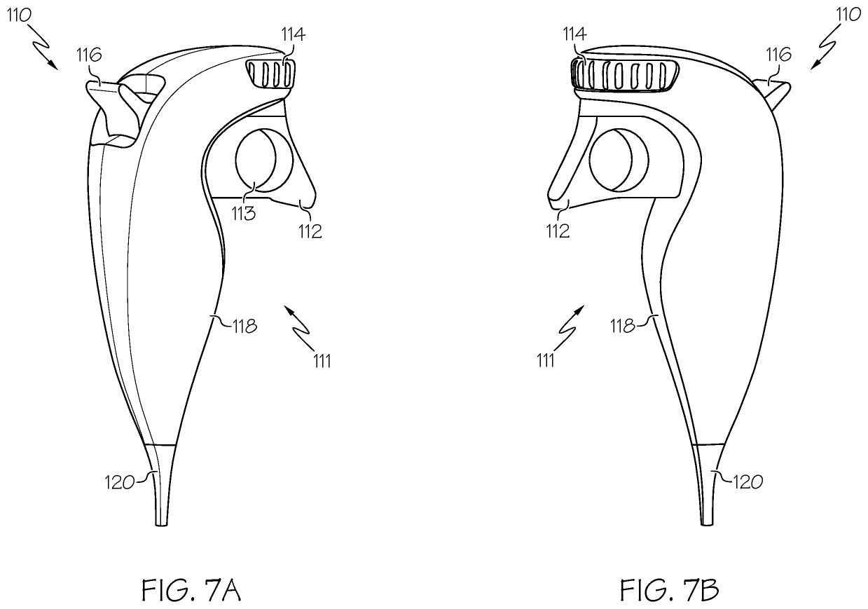

FIGS. 7A and 7B show an alternate embodiment of a medical device 110. Medical device 110 includes a handle 111 having a handle body 118 and three actuators: lever 116, knob 114, and trigger 112. Strain relief 120 provides the junction between handle 111 and the device sheath (not shown). Lever 116 may control the articulation direction of distal end 1030, exemplified in FIG. 1. For example, pushing lever 116 away (or up as shown in the Figures) can result in the left articulation of the distal end, and pulling lever 116 down may result in the right articulation of the distal end. Alternate actions are within the scope of this disclosure. For example, pushing lever 116 away can result in the right articulation of the distal end, and pulling lever 116 down can result in the left articulation of the distal end. The device can also be articulated up or down in a similar fashion.

Knob 114 is located on the forward face of handle 111, facing away from the user during operation. A plan in which knob 114 rotates is perpendicular to the center axis of device 110 that extends through handle 118, strain relief 120, and the device shaft. Knob 114 can control the rotational movement of the end effector 1025, exemplified in FIG. 1. For example, rotating knob 114 to the left can translate to rotation of the end effector in a counterclockwise motion relative to the device shaft, and rotating knob 114 to the right can translate to rotation of the end effector in a clockwise motion relative to the shaft. Alternate actions are within the scope of this disclosure. For example, rotating knob 114 to the right can translate to the rotation of the end effector in a counterclockwise motion, and rotating knob 114 to the left can translate to rotation of the end effector in a clockwise motion.

Trigger 112 may control the actuation (e.g. open/close movement) of the end effector 1025, exemplified in FIG. 1. For example, initiating trigger 112 (pulling trigger 112 into handle body 118) can translate to opening the end effector (not shown), and releasing trigger 112 (allowing or pushing trigger 112 out of body 118) can translate to closing the end effector. Alternate actions are within the scope of this disclosure. For example, actuating trigger 112 can translate into opening the end effector, and releasing trigger 112 from handle 118 can translate into closing the end effector.

Handle 111 may contain any combination of the trigger 112, knob 114, and lever 116. For example, handle 11 may only contain knob 114 and trigger 112, or any other combination. Further, each actuator is not limited to the described action. For example, trigger 112 can control the articulation of distal end 1030 (shown in FIG. 1), knob 114 can control the open/close movement of end effector 1025 (shown in FIG. 1), and knob 116 can control the rotation of the distal tip (shown in FIG. 1). Any combination thereof is also permissible.

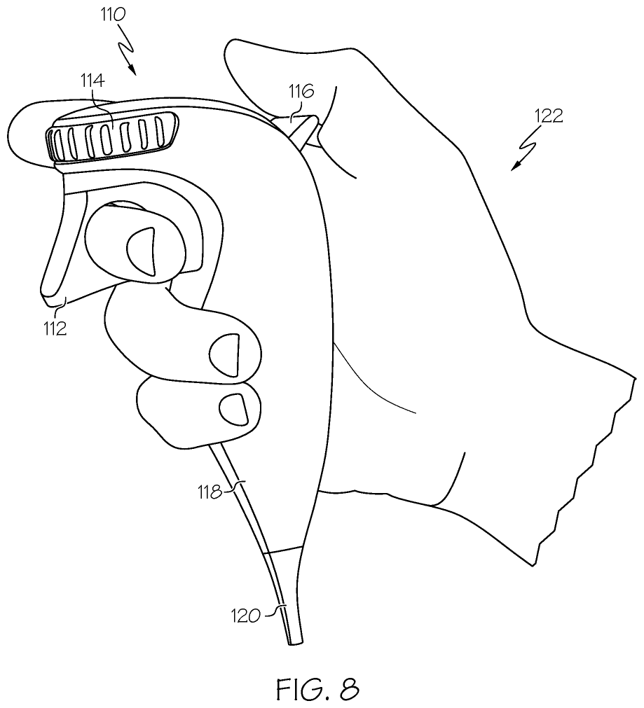

FIG. 8 shows how a user 122 may grip medical device 110. The thumb of user 122 may control lever 116. The middle finger of user 122 may fit within an opening (a slot 113) of trigger 112 to control the actuation (e.g. open and closing) of the distal tip of medical device 110, and the index finger of user 122 may control the rotation of knob 114. The remaining fingers and palm of user 122 may grip handle body 118. Thus, in use, trigger 112 and knob 114 face away from user 122, lever 116 faces towards user 122, and knob 114 is above trigger 112. In addition, trigger 112, knob 114, and lever 116 lie in a common plane.

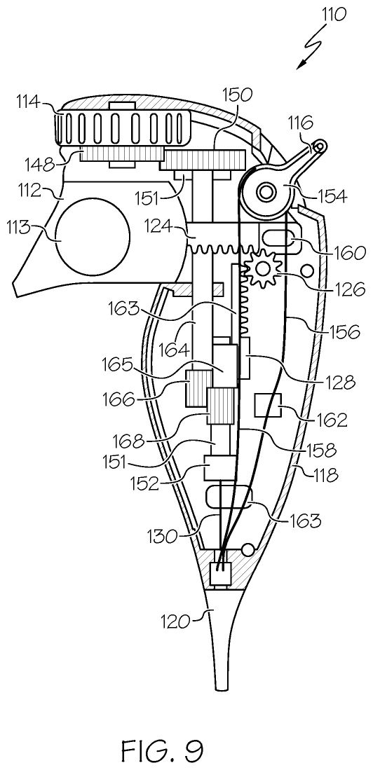

FIG. 9 shows a cross-section of handle 110 of FIGS. 7A and 7B. Knob 114 may control the rotational movement of the distal tip relative to a shaft of the medical device. Knob 114 is fixedly coupled to pinion gear 148. A plurality of extensions and indentions of pinion gear 148 mesh with the extensions and indentions of a pinion gear 150. Pinion gear 150 is fixedly coupled to a proximal end of a shaft 164. Pinion gear 150 and shaft 164 may be held in an upright position by an extension 151 of handle body 118. Pinion gear 150 may sit above extension 150 and shaft 164 may extend through extension 150. A pinion gear 166 may be fixedly coupled to a distal end of shaft 164. The plurality of extensions and indentions of pinion gear 166 mesh with the extensions and indentions of a pinion gear 168. Pinion gear 168 may be fixedly coupled to the proximal end of a shaft 151. Ferrule 152 may be coupled to the distal end of shaft 151 and may be fixedly connected to a control wire 130. Control wire 130 may be fixedly coupled to ferrule 152 by adhesives, crimps, or any alternative coupling methods commonly known in the art. A distal end of wire 130 (not shown) may be coupled to a distal assembly/end effector at a distal end of device 110. When knob 114 is rotated, pinion gear 148 is rotated consequently. Thus, due to the intermeshing of the extensions and indentions of pinion gear 148 with pinion gear 150, when pinion gear 148 is rotated, pinion gear 150 is subsequently rotated in the opposite direction. This movement consequently rotates pinion gear 166, which is coupled to the distal end of shaft 164. Due to the meshing of the extensions and indentions of pinion gear 166 with pinion gear 168, when pinion gear 166 is rotated, pinion gear 168 rotates in the opposite direction. Because shaft 151 is coupled to pinion gear 168, when pinion gear 168 is rotated, shaft 151 and ferrule 152 may rotate, accordingly. This movement translates to the rotation of the distal tip assembly/end effector relative to then shaft of medical device 110. In addition, due to the arrangement of the parts in the handle body 118, clockwise rotation of knob 114 results in clockwise rotation of wire 30, and vice versa.

Still referring to FIG. 9, lever 116 includes a cam 154. Control wires 156 and 158 are coupled to cam 154 such that, when lever 116 is pushed up, cam 154 is rotated counterclockwise and tension is created in steering wire 156. When lever 116 is pulled down, cam 154 rotates clockwise, creating tension in steering wire 158. The tension created in steering wires 156 and 158 translates to articulation at the distal tip, since wires 156, 158 are coupled to the distal tip (e.g. the articulation joint). Control blocks 128, 160, 162, and 163 of handle body 118 control the location of steering wires 156 and 158 in the handle and assist in guiding the wires 156, 158 in the handle body 118 as the wires 156, 158 translate.

Trigger 112 includes a slot 113 that enables a user to use a finger to actuate trigger 112. Slot 113 may be of many shapes and sizes to enable the user to actuate trigger 112. For example, the slot may be circular, as shown in FIG. 9, ovular, rectangular, or any shape to enable a user to use a finger to actuate trigger 112. Rack 124 is fixedly coupled to trigger 112. When trigger 112 is actuated (pulled into handle body 118 via a slot within handle body 118), rack 124 is translated longitudinally into the handle, and towards the user. The extensions and indentions of rack 124 mesh with the indentions and extensions of pinion gear 126. When rack 124 is translated longitudinally into the handle, pinion gear 126 rotates clockwise. This rotation of pinion gear 126 results in the lateral (up and down; proximal and distal) movement of a rack 163. For example, when pinion gear 126 is rotated clockwise, rack 163 is raised (moved proximally). Similarly, when pinion gear 126 is rotated counterclockwise, rack 163 is lowered (moved distally). Rack 163 may be fixedly coupled to a shaft 165 by adhesives, welding, or any other means commonly known in the art. Shaft 165 is coupled to the proximal end of a pinion gear 168. As previously described, pinion gear 168 is coupled to shaft 151 and ferrule 152 such that, when pinion gear 168 is raised or lowered, shaft 151 and ferrule 152 move accordingly. This movement translates to actuation (e.g. open and close movement) of the distal tip/end effector of medical device 110.

The device of FIGS. 7A-7B is used in a similar way as device 10 of FIGS. 2A-2B. To do so, the user may first introduce the distal end of device 110 into a GI tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens or organs reachable via the GI tract, any other natural opening or body tract, bodily incision, or through a delivery device, such as an endoscope or sheath. Once the desired site is accessed, the user can actuate one or more actuators, including knob 114, lever 116, and trigger 112, with only one hand (or both hands if desired), to control the articulation of the distal end, the actuation of the end effector, and/or the rotation of the end effector relative to the shaft of medical device 110.

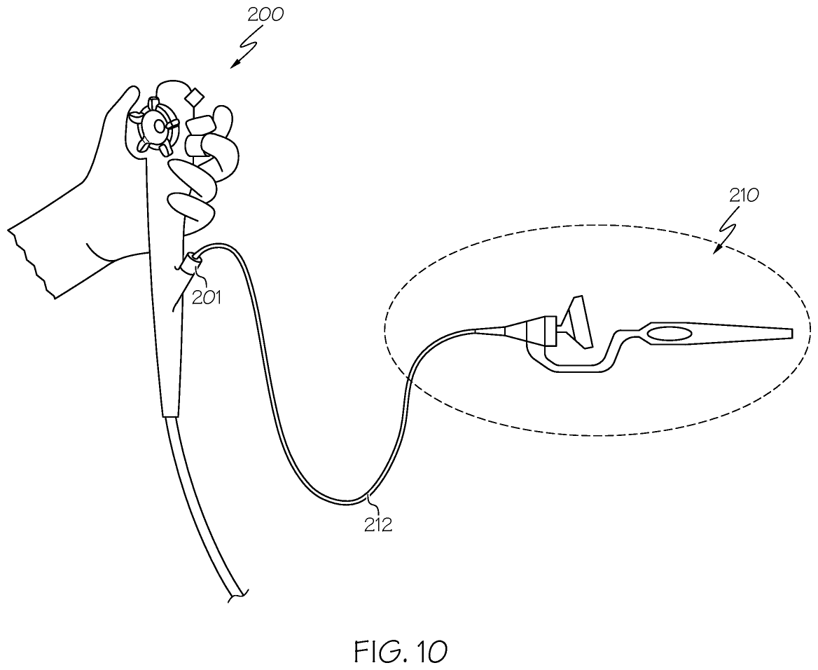

FIG. 10 depicts an alternate embodiment of a medical device 210. As shown in FIG. 10, a shaft 212 of medical device 210 may be inserted in a scope 200 through port 201.

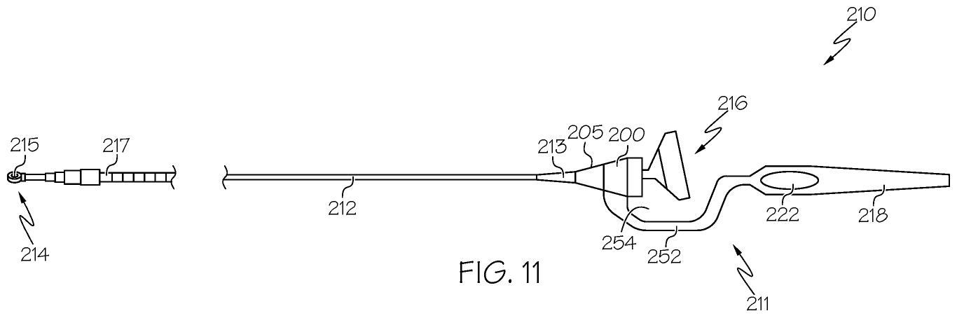

FIG. 11 shows medical device 210, which includes a handle 211, an actuator base 205, an actuator assembly 216, shaft 212, and an articulation zone 217 and an end effector 215 at a distal end 214. Actuator assembly 216 may include a knob, joystick, or any actuator commonly known in the art. End effector 215 may be comprised of any end effector commonly known in the art, such as graspers, baskets, brushes, scissors, forceps, knives, etc. End effector 215 is coupled to the distal-most end of articulation zone 217, which enables device 210 to be moved in multiple directions by mechanisms that are to be described further herein. The proximal end of articulation zone 217 is coupled to the distal end of shaft 212. A strain relief 213 is coupled to the proximal end of shaft 212. Strain relief 213 serves as a junction between shaft 212 and actuator base 205.

Handle 211 includes a proximal extension 218, a slot 222, a U-shaped extension 252, and an extension 200. Handle 211 may be comprised of any rigid material. Extension 200 includes an opening to permanently or temporarily receive and couple with handle base 205 by any means commonly known in the art, including a press fit, adhesives, snap-fit, etc. Handle 211 may be coupled to actuator base 205 such that, when handle 211 is rotated about its longitudinal axis, medical device 210 rotates in the same or similar manner. Actuator assembly 216 is internally constrained within actuator base 205 and includes a ball-and-socket joint, to be described further herein. Additionally, the connection of handle 211 to actuator base 205 creates a gap 254 between handle 211 and actuator assembly 216. Gap 254 enables a user to comfortably grip actuator assembly 216 without interference from handle 211. Extension 218 extends from the proximal end of U-shaped extension 252. Extension 218 may be of a sufficient length to enable a user to fully grasp the extension with one hand. Extension 218 may be many shapes and sizes. For example, extension 218 may taper towards the proximal end for a more comfortable grip (as shown), and extension 218 may be circular or rectangular in a cross-sectional view. Located on or adjacent to the distal end of extension 218, slot 222 can be many shapes and sizes to enable a user to insert one or more fingers through slot 222. For example, slot 222 can be ovular, as shown, circular, square, rectangular, etc.

The handle is configured such that a user (not shown) may grip the handle with a single hand in a number of ways. For example, the user may grip actuator assembly 216 with one or two fingers and a thumb (or only two fingers, not including the thumb) from the single hand. Remaining fingers may grip extension 218 by wrapping around the extension and holding the extension against the palm of the single hand. One or multiple fingers from the single handle may be inserted through slot 222, as well. Handle 211 may then be rotated such that the handle is in a more neutral or natural position for the user relative to the actuator assembly 216. For example, handle 211 may be held such that the U-shaped extension is below the actuator assembly 216, as shown, or to either side of actuator assembly 216.

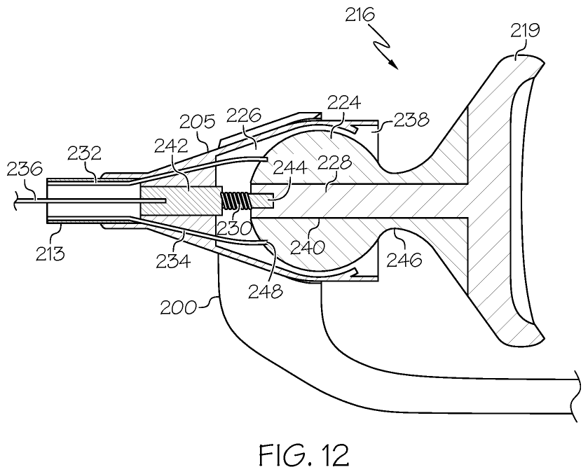

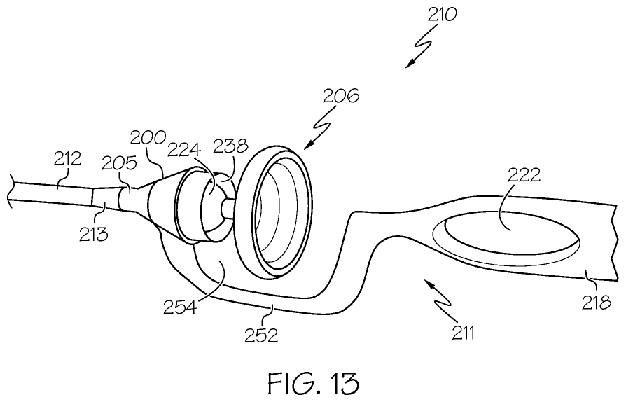

FIG. 12 is a cross-sectional view of strain relief 213, actuator base 205, and actuator assembly 216, and FIG. 13 shows a magnified perspective view of actuator assembly 216 and a distal portion of handle 211 of medical device 210. Actuator assembly 216 includes an actuator 219 and a ball 224. Extension 228 extends from a proximal portion of actuator 219 through ball 224, along a center axis. Extension 228 is movable within ball 224 along a longitudinal axis of extension, as will be described further herein. Ball 224 is constrained by a socket 238 such that ball 224 cannot be removed from actuator base 205, but can be moved (rotated about its center) relative to actuator base 205, as actuator 219 is moved in any orientation (e.g. left, right, up, down, or any combination of those movements).

Still referring to FIG. 12, a control wire 236 extends through shaft 212 and strain relief 213 and is fixedly coupled to the distal end of a block 242. A spring 230, or other flexible connector, extends from the proximal end of block 242 along the center axis and from the distal end of a block 244 along the center axis. Block 244 is fixedly coupled to extension 228 along the center axis of extension 228, for example via a screw connection, press fit, welding, adhesive, etc. Extension 228 extends through the center axis of ball 224. Steering wires 232, 236 extend from the distal articulation zone 217 of shaft 212, through shaft 212, and may be fixedly coupled to ball 224 on opposite sides of ball 224. Medical device 210 is not limited to contain two steering wires 232, 236 (as shown) but may contain one or several steering wires, depending on the desired articulation of the distal tip. For example, two steering wires may control the articulation direction in two directions (e.g. left, right), whereas four steering wires may control the articulation direction of the distal tip in four directions (e.g. left, right, up, down).

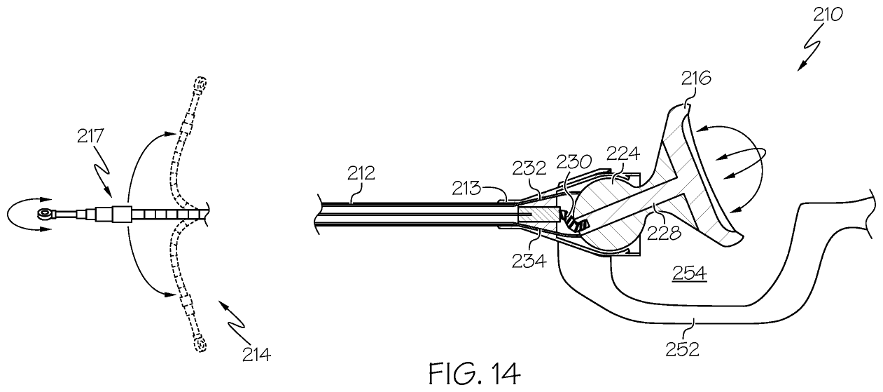

FIG. 14 shows a partial cross-sectional view of device 210 during articulation. Actuator assembly 216 may be moved in any direction (as described above), putting tension on one or more steering wires, such as steering wires 232, 234. During rotation of ball 224, spring 230 flexes, as shown in FIG. 14. The rotation of ball 224 translates to articulation at the distal tip 214. For example, when actuator assembly 216 is moved upwards, tension is placed on steering wire 234. This tension results in the downwards movement of articulation section 217. Similarly, when actuator assembly 216 is moved downwards, tension is placed on steering wire 236, resulting in the upward movement of distal end 214.

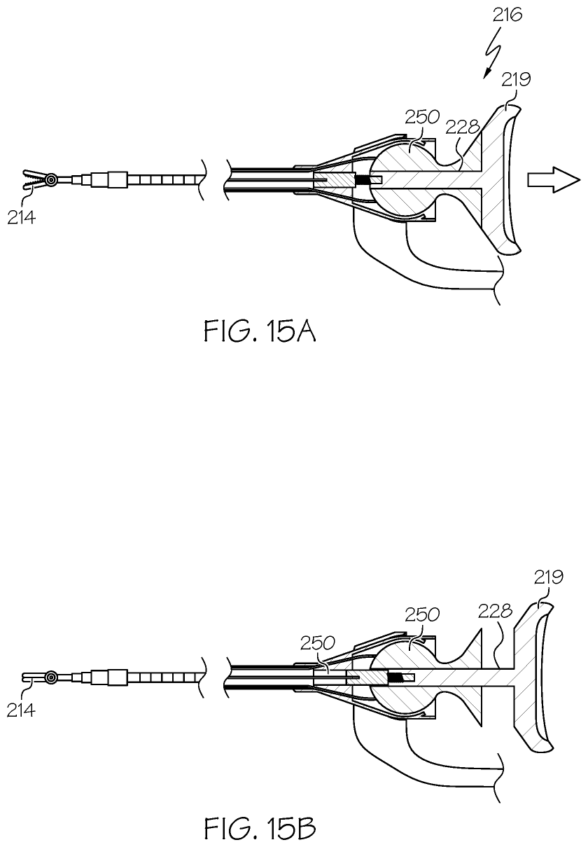

FIGS. 15A and 15B show a cross-sectional view of handle 210 when the distal tip 214 actuated, e.g. opened or closed. When an end effector of distal tip 214 is open, the distal end of actuator 219 is pressed against a proximal wall of ball 224. Extension 228 extends from the distal wall of actuator 219 through the center axis of ball 224. When the end effector of distal tip 214 is closed, actuator 219 is configured to pull away from the proximal face of ball 224. Extension 228 can be pulled along the center axis of ball 224. This movement also pulls spring 230, block 242, and control wire 236 along the center axis of ball 224. This movement translates to the closure of the end effector of distal tip 214. Pushing actuator 219 back into ball 224 may actuate or open the end effector of distal tip 214. Actuator 219 may also be configured such that, when actuator 219 is rotated relative to ball 224, the end effector of distal tip 214 may rotate relative to shaft 212. For example, if actuator 219 is rotated in a clockwise direction, distal tip 214 is rotated clockwise, accordingly, and vice versa.

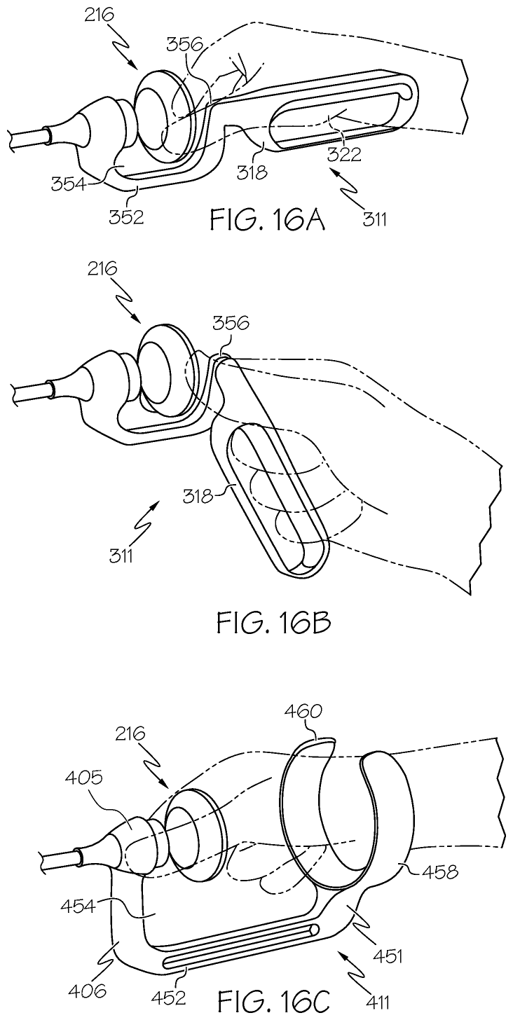

FIGS. 16A, 16B, and 16C depict alternative handle embodiments and how a user may grip these handles. These handles may be used with the actuator assembly 216 of FIGS. 11-15B, and coupled to actuator assembly 216 in the same manner as described above. Only differences in the handles will be described.

Referring to FIG. 16A, a handle 311 includes a handle extension 318 having a slot 322 therein. A U-shaped extension 352 is pivotally attached to a distal end of extension 318. A gap 354 is defined between actuator assembly 216 and U-shaped extension 352. A pivot pin 356 is located between the proximal end of U-shaped extension 352 and distal end of handle extension 318. In the state of FIG. 16A, handle extension 318 and handle slot 322 is in the same plane as U-shaped extension 352. A user 322 may grip actuator assembly 216 using an index finger and thumb. The remaining fingers of user 322 may grip handle extension 318 or extend through finger slot 322.

FIG. 16B shows handle 311 in a pivoted state. In this state, handle 318 is perpendicular to the U-shaped handle extension 352. A user 322 may grip actuator assembly 216 using an index finger and thumb. The remaining fingers of user 322 may grip handle extension 318 and/or extend through finger slot 322. This pivoted state of handle 311 may enable a user to hold the handle in a more neutral grip.

FIG. 16C shows another embodiment of a handle 411. Handle 411 includes inwardly curved extensions 458 and 460 that lie in a plane perpendicular to, or approximately perpendicular to, a longitudinal axis of the medical device. Extensions 458, 460 also extend up from the proximal end of a handle extension 451. Extensions 458, 460 are curved and have a space between their tops to allow for insertion of a user's wrist into a space defined between extensions 458, 460. Handle extension 452 extends from the distal end of handle extension 451 and is fixedly coupled to (or integral with) a handle extension 406. Handle extension 406 extends downward from handle base 405. Handle extensions 406, 452, 451 are configured in a U-shaped formation to create a gap 454 between the actuator assembly 216 and the handle 411. Gap 454 enables a user to grip actuator assembly 216 without interference from handle 411. A user may grip actuator 216 in a same or similar configuration, as described in previous embodiments. For example, a user may grip actuator assembly 216 using an index finger and thumb. The remaining fingers of the user may be configured in any orientation comfortable for the user. The user may insert a hand through extensions 458, 460 such that the extensions rest on a user's wrist or a distal portion of the arm, or insert the user's wrist/arm through the upper gap between the ends of extensions 458, 460, to place the wrist/arm within the circular space between extensions 458, 460. Slot 452 may be for ornamental purposes.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

Accordingly, various aspects discussed herein may help to improve the efficacy of treatment, for example, a procedure to treat a treatment site. Various aspects discussed herein may help to reduce and/or minimize the duration of the procedure, may reduce the risks of inadvertent manipulation by the user, and/or may help reduce risks of inadvertent contact with tissue or other material during delivery, repositioning, or usage of a medical device in the procedure.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

What is claimed is:

1. A medical device, comprising:
a handle having at least one actuator;
a shaft having a proximal end and a distal end, the proximal end connected to the handle; and
a distal assembly connected to the distal end of the shaft, the distal assembly including an end effector,
wherein the handle is configured so that a single hand of a user can operate the at least one actuator to (1) actuate the end effector, (2) rotate the end effector relative to the shaft, and (3) articulate a distal portion of the shaft.

2. The medical device of claim 1, wherein the at least one actuator includes a first actuator, a second actuator, and a third actuator, wherein the first actuator rotates the end effector relative to the shaft, the second actuator actuates the end effector, and the third actuator articulates the distal portion of the shaft.

3. The medical device of claim 2, wherein at least the first actuator is a trigger, the second actuator is a knob, and the third actuator is a knob, wherein the handle is configured such that the user may simultaneously place an index finger of the single hand on the trigger, a thumb of the single hand on the first knob, a middle finger of the single hand on the second knob, and a palm of the single hand against the handle.

4. The medical device of claim 3, wherein the handle is configured such that, in use, the first knob is facing away from the user, the second knob is on the left of the handle body relative to the user, and the trigger is on the top of the handle.

5. The medical device of claim 1, wherein a mechanism to control the actuation of the end effector includes a first rack, a pinion gear meshed with the first rack, and a second rack meshed with the pinion gear and coupled to a control wire.

6. The medical device of claim 5, wherein, when the first rack is moved into and out of a handle body of the handle, the pinion gear rotates clockwise and counterclockwise respectively, moving the second rack and the control wire proximally and distally respectively.

7. The medical device of claim 1, wherein a mechanism to control the rotation of the end effector relative to the shaft includes a first pinion gear, and a second pinion gear meshed with the first pinion gear, wherein the control wire is within the second pinion gear such that, when the first pinion gear is rotated clockwise and counterclockwise, the second pinion gear rotates counterclockwise and clockwise respectively, thereby rotating the control wire.

8. The medical device of claim 1, wherein a mechanism to control the articulation of the distal portion of the shaft includes a first control wire and a second control wire coupled to a pulley such that, when the pulley is rotated clockwise, the first control wire is pulled into tension and, when the pulley is rotated counterclockwise, the second control wire is pulled into tension.

9. The medical device of claim 2, wherein the first actuator is a knob, the second actuator is a trigger, and the third actuator is a lever, and wherein the knob, the trigger, and the lever are configured such that the user may simultaneously place an index finger of the single hand on the knob, a thumb of the single hand on the lever, a middle finger of the single hand on the trigger, and a palm of the single hand against the handle.

10. The medical device of claim 9, wherein the handle is configured such that, in use, the knob is facing away from the user, the lever is facing towards the user, and the trigger is facing away from the user.

11. The medical device of claim 1, wherein a mechanism to control the actuation of the end effector includes of a first rack, a pinion gear meshed with the first rack, and a second rack meshed with the pinion gear and coupled to a control wire.

12. The medical device of claim 11, wherein, when the first rack is moved into and out of a handle body of the handle, the pinion gear rotates clockwise and counterclockwise respectively, moving the second rack and the control wire proximally and distally respectively.

13. The medical device of claim 1, wherein a mechanism to control the rotation of the end effector relative to the shaft includes a first pinion gear, a second pinion gear meshed with the first pinion gear and fixedly coupled to a proximal end of a shaft, a third pinion gear fixedly coupled to a distal end of the shaft, and a fourth pinion gear meshed with the third pinion gear, wherein the control wire is within the fourth pinion gear such that, when the first pinion gear is rotated clockwise and counterclockwise, the fourth pinion gear rotates clockwise and counterclockwise respectively, thereby rotating the control wire.

14. The medical device of claim 1, wherein a mechanism to control the articulation of the distal portion of the shaft includes a first control wire and a second control wire coupled to a cam such that, when the cam is rotated clockwise, the first control wire is pulled into tension, and, when the cam is rotated counterclockwise, the second control wire is pulled into tension.

15. The medical device of claim 1, wherein the at least one actuator is one actuator, and the one actuator is a knob coupled to a base of the handle by a ball at the distal end of the knob and a socket on the proximal end of the base of the handle, and wherein the knob is configured to include:
a first mechanism to control the actuation of the end effector and including a control wire coupled to a distal end of the knob such that, when the knob is pulled proximally and pushed distally, the control wire is translated accordingly;
a second mechanism to control the rotation of the end effector relative to the shaft and including the control wire coupled to a distal end of the knob such that, when the knob is rotated clockwise and counterclockwise, the control wire is rotated clockwise and counterclockwise, respectively; and
a third mechanism to control the articulation of the distal portion of the shaft and including a first articulation wire and a second articulation wire coupled to the ball and configured such that, when the knob is moved in a first direction, the first articulation wire is pulled into tension, and, when the knob is moved in a second direction opposite the first direction, the second articulation wire is pulled into tension.

16. A medical device, comprising:
a handle having one actuator;
a shaft having a proximal end and a distal end, the proximal end connected to the handle; and
a distal assembly connected to the distal end of the shaft, the distal assembly including an end effector,
wherein the handle is configured so that a single hand of a user can operate the one actuator to (1) actuate the end effector, (2) rotate the end effector relative to the shaft, and (3) articulate a distal portion of the shaft.

17. The medical device of claim 16, wherein the one actuator is a knob coupled to the handle by a ball and a socket joint.

18. The medical device of claim 16, wherein the handle is separable from the actuator.

19. A method of operating a medical device, comprising:
positioning the medical device inside a body lumen, wherein the medical device includes a handle having at least one actuator, a shaft having a proximal end and a distal end, the distal end being connected to the handle, and a distal assembly connected to the distal end of the shaft, the distal assembly including an end effector; and
articulating a distal portion of the shaft with the at least one actuator using a single hand;
rotating the end effector relative to the shaft with the at least one actuator using the single hand; and
actuating the end effector with the at least one actuator using the single hand.

20. The method of claim 19, wherein the at least one actuator is three actuators configured such that a user can simultaneously contact the three actuators at a same time using the single hand.
